(19) United States
(12) Patent Application Publication
(10) Pub. No.: US 2011/0245609 A1
(43) Pub. Date: Oct. 6, 2011

(54) VIDEO ADAPTER FOR LARYNGOSCOPE

(76) Inventor: Vadim LASER, Maineville, OH (US)

(21) Appl. No.: 13/049,030

(22) Filed: Mar. 16, 2011

Related U.S. Application Data
(60) Provisional application No. 61/318,819, filed on Mar. 30, 2010.

(51) Int. Cl. A61B 1/04

(52) U.S. Cl. 600/109

(57) ABSTRACT

A portable, low power, low cost, video adapter for a common laryngoscope, having a slim self-illuminating CMOS camera, attachable with a disposable bracket to the laryngoscope blade and connected via flexible wire to the miniature battery powered color LCD monitor that is detachably mounted either on the wrist band or on top of laryngoscope handle.

### CROSS-REFERENCE TO RELATED APPLICATIONS

This application claims the benefit of priority to U.S. provisional application 61/318,819, filed Mar. 30, 2010, the disclosure of which is incorporated by reference.

### FIELD OF THE INVENTION

This invention is concerned with an imaging device used for assisting emergency medical service (EMS) personal and other medical practitioners during performance of an endotracheal intubation procedure.

### BACKGROUND OF THE INVENTION

Endotracheal intubation is a commonly used procedure during which a semi rigid plastic tube, called an endotracheal tube, is inserted into a patient's trachea through the open mouth. Typically, a laryngoscope is used to view the throat area through the open mouth while the tip of the endotracheal tube advances toward the vocal cords. This procedure is not a trivial one especially when it needs to be performed in the inconvenience of emergency type situations and with the patients having various irregularities and complications in the throat and neck areas. Use of intubation assisting tools is important to the first response paramedics, who often save lives of traumatized and unconscious victims by quickly administering the endotracheal tube into the trachea. Extra minutes and even seconds of delay can be fatal.

During the last decade, many researches and physicians advocated use of endoscopes during the intubation procedures. The tip of the flexible shaft of endoscope is inserted inside the endotracheal tube and temporarily secured near the distal end of it. Alternatively, the endoscope or a small camera can be inserted in the mouth alongside and/or with the endotracheal tube. Depending on the type of an endoscope used, a practitioner performing intubation can see the area in the immediate vicinity of the distal end of the endotracheal tube either on the video screen of a monitor or looking in the ocular of a simpler endoscope. It is obvious, that such use of an endoscope reduces the number of traumas and helps to ease and speed up the intubation process. Many existing endoscopes can be used for such application. However, a typical medical endoscope is an expensive and bulky instrument, utilizing powerful light sources and fiber optic bundles for delivery of light and/or images.

Often two paramedics are involved trying to resuscitate an unconscious victim. While one works on the chest of the affected person, the other tries to intubate. In such situations, there is not even a good place to position the video monitor for observation of images sent by the endoscope or a camera. The endoscopes currently known are rather bulky and expensive. High price and inconvenient construction often preclude use of the endoscopes in the battlefield, on the streets and other ambulance situations. There is a need for a portable and inexpensive device for assistance in performing intubation, especially in the demanding conditions of emergencies

Thus, there remains a need for further improvements in portable imaging-type intubation assistance tools.

### SUMMARY OF THE INVENTION

The present invention provides a portable video adapter for a laryngoscope that is small in size and light in weight, self-powered and suitable for assisting endotracheal intubations.

The invention also provides a portable video adapter for laryngoscope that has low cost.

The invention further provides a portable video adapter for a laryngoscope with an easily detachable and replaceable probe, capable of being disinfected for multiple uses.

The invention still further provides a portable video adapter for a laryngoscope that has inherently high reliability due to absence of components that are typically or statistically vulnerable to failure.

The invention also provides a portable video adapter for compatible use with an existing laryngoscope, which can already include an illuminator including a fiber optic illuminator or a standard (light bulb or light emitting diode (LED)) illuminator, the existing laryngoscope already in the possession of a medical practitioner who performs intubations.

The invention further provides a portable video adapter for a laryngoscope having an improved field of view without sacrificing image quality.

The present invention provides a portable video adapter for a laryngoscope suitable for use during endotracheal intubation comprising: a slim imaging probe, a probe clip attachable to the laryngoscope blade, a miniature high resolution color video monitor that can be conveniently mounted either on the wrist or on a portion of the laryngoscope, such as the top of the laryngoscope handle.

The present invention also provides a swivel unit as a part of the monitor mounting feature for quick directional alignment of the monitor screen.

The present invention also provides a disposable, inexpensive probe securement or clip that allows the same imaging probe to be used with a variety of existing permanent or disposable laryngoscope blades.

### BRIEF DESCRIPTION OF THE DRAWINGS

FIG. 1 shows the portable video adapter system of the present invention including a slim probe attached to a laryngoscope blade.

FIG. 2 shows the portable video adapter system of FIG. 1, the display monitor secured on the wrist of the hand of a person performing the intubation.

FIG. 3 shows a cross-sectional view of the internal construction of the slim probe.

FIG. 3a shows expanded view of the imaging head of the probe shown in FIG. 3.

FIG. 4 shows a conventional laryngoscope blade of Macintosh type with an integral lamp.

FIG. 5 shows the conventional laryngoscope blade of FIG. 4 with the probe clip attached.

FIG. 5A shows a cross-sectional view of the laryngoscope blade and probe clip of FIG. 5, taken through line A-A.

FIG. 6 shows the laryngoscope blade with the slim probe fitted into the attached probe clip.

FIG. 6A shows a cross-sectional view of the laryngoscope blade and probe clip of FIG. 6, taken through line B-B.

FIG. 7 shows a side view of the display monitor affixed to a flexible wrist band, having a swivel device providing freedom for vertical angular adjustment (roll angle).

FIG. 8 shows a plan view of the display monitor of FIG. 7 providing freedom for horizontal angular adjustment.

FIG. 9 shows a cross section side view of the display monitor with the attached swivel unit and support plates equipped with reclosable fasteners.

FIG. 10 shows a side view of the display monitor removably affixed to the flexible wrist band through the swivel device and the reclosable fasteners, providing increased freedom for vertical angular adjustment.

### DETAILED DESCRIPTION OF AN EMBODIMENT OF THE INVENTION

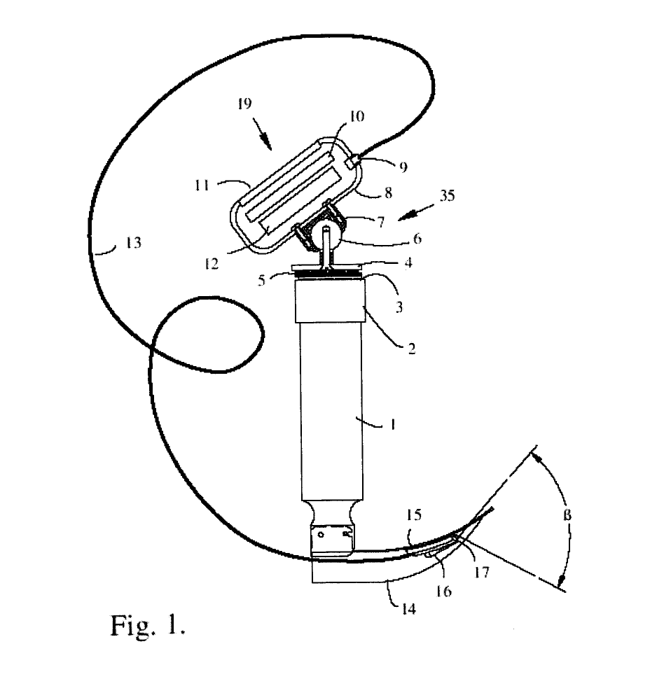

FIG. 1 illustrates how components of the portable video adapter system are interconnected. The slim endoscope probe 17 is attached to the laryngoscope blade 14 by means of a securement, illustrated as a clip 15, next to an integral light source 16 of the laryngoscope blade. The bracket can include an elongated U-shaped channel having a base and opposed side walls. When the probe is inserted in the channel, it is securely held there by the walls, which normally are inclined towards each other and pushed apart by the body of the probe. Other type of the bracket can include a removable adhesive strip inside the channel. A probe-holding clip can be made as a part of the laryngoscope blade. Depending on type of the laryngoscope, the light source 16 can be either a bulb, or an end of the fiber bundle delivering light from a bulb residing inside the laryngoscope handle 1. A portable video display monitor 19 is provided, mounted on the top of the cup 2 of the laryngoscope handle 1. A small diameter flexible cable 13 electrically connects the slim endoscope probe 17 with the portable monitor 19.

The slim endoscope probe 17 is configured to provide a wide field of view β, which facilitates quick finding of the vocal chords that represent entrance into the trachea. The cable 13 is attached and sealed, preferably permanently, at one end to the rear end of the endoscope probe 17. On the opposite end, the cable 13 is attached to a miniature, sealed connector 9, which electronically connects the slim probe 17 to the video monitor 19. The video monitor 19 has a display panel 11, typically a liquid crystal display (LCD) type display or an organic light emitting diode (OLED) type display. Inside the monitor enclosure 8 that fixes the display panel 11, there are electronics board 10 that include the display drivers, power supply circuits and the communication electronics, and a rechargeable battery 12. The cable 13 delivers power and control signals to the endoscope probe 17, and returns to the monitor the signals representing a video image.

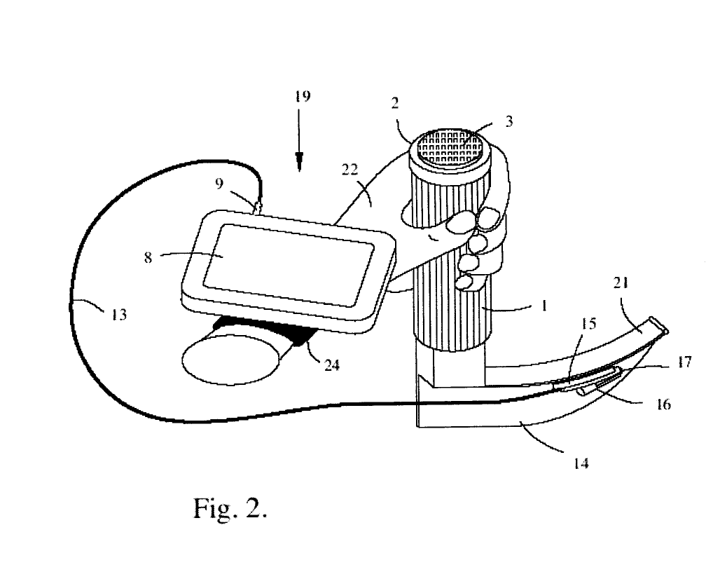

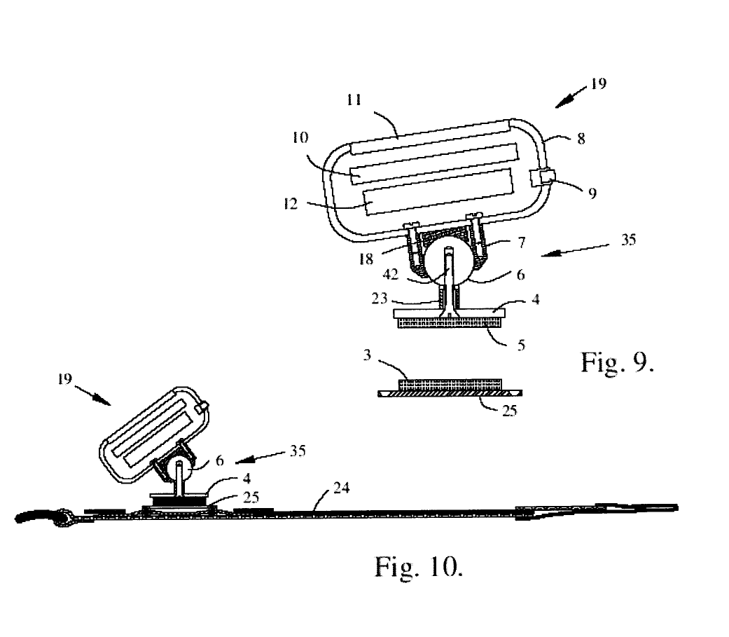

The video monitor 19 is mounted by means of a swivel unit 35. Detailed construction of an embodiment of the swivel unit is illustrated on FIG. 9. A foot 4 of the illustrated swivel unit is equipped on its bottom surface with a reclosable fastener, such as an interlocking mushroom-type fastener array 5, including one described in U.S. Pat. Nos. 5,077,870, 5,868,987, 6,470,540, 6,592,800, and 7,275,290, the disclosures of which are incorporated herein by reference in their entirety. A pivot joint, shown as a plastic ball 6, is attached to the foot 4 by means of a screw 42 and a spacer 23. The ball 6 resides inside a socket, defined by a cylindrical ball holder 7, and is partially exposed through the opening in the holder 7. The ball holder 7 is attached to the back side of the enclosure 8 of the video monitor 19. A compressed elastic material, illustrated as spacer 18 between the ball 6 and the enclosure 8, provides a friction force to the ball that prevents it from free rotation, while allowing manual angular adjustment of the angular position of the monitor 19. A second reclosable fastener 3 is applied to the top of the cup 2 of the laryngoscope handle 1. This allows the portable video monitor 19 to be snappily and removably mounted on the top of the laryngoscope. A similar pad of reclosable fastener material 3 is attached to a thin plate 25, typically made of stainless steel, as shown on FIG. 9. The plate 25 is fastened mechanically or adhesively to a wrist band 24. This allows the video monitor 19 to be snappily fastened to and removed from the wrist band 24. FIG. 2 illustrates how the video adapter system is configured with the video monitor 19 residing on the wrist 22 of the medical practitioner. Also illustrated is the reclosable mushroom fastener 3 attached to the top of cup 2 of the laryngoscope handle 1. As needed, the video monitor can be instantly relocated from the user's wrist 22 to the laryngoscope handle 1, and back.

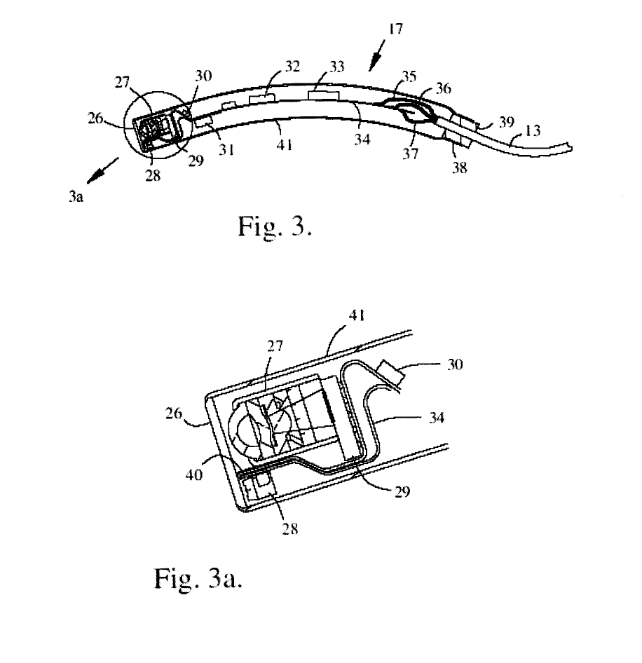

The FIG. 3 shows a longitudinal cross section of the slim endoscope video probe 17. The distal end C of the probe 17 is also shown in expanded view in FIG. 3a. Inside the tubular enclosure 41 and behind an optically clear window 26, and secured at one end of the tubular enclosure 41, there are imaging components: a video sensor 29, a wide angle lens 27, and an illuminating LED 28 mounted on a thin flexible electrical circuit 34. The sensor 29 includes a CMOS camera as described in US Publication 2008-0091064, the disclosure of which is incorporated herein by reference in its entirety. There is a light separator 40, which prevents strain light coming into the imaging lens 27 directly from the LED 28 or reflected from the surfaces of the window 26. Other embodiments of the imaging components in the distal end of the probe are described in the aforementioned and incorporated US Publication 2008-0091064. The portion of the flex circuit 34 that extends further inside the tubular enclosure 41 carries other electronic components 30, 31, 32 and 34, such as a noise reducing bypass capacitors, an oscillator and a microprocessor. The small diameter flexible cable 13 enters the proximal end 38 of the tubular probe 17 through an insert 39, such as a rubber or elastomeric material. Crimping of the tubular end 38 compresses the insert 39, thus providing a moisture-proof seal and a strong mechanical assembly. The tubular enclosure 41 of the probe 17 may be straight to fit the straight laryngoscope blades such as Miller type, or curved as shown in FIG. 3 in order to fit the curvature of curved blades such as the Macintosh type laryngoscope blades.

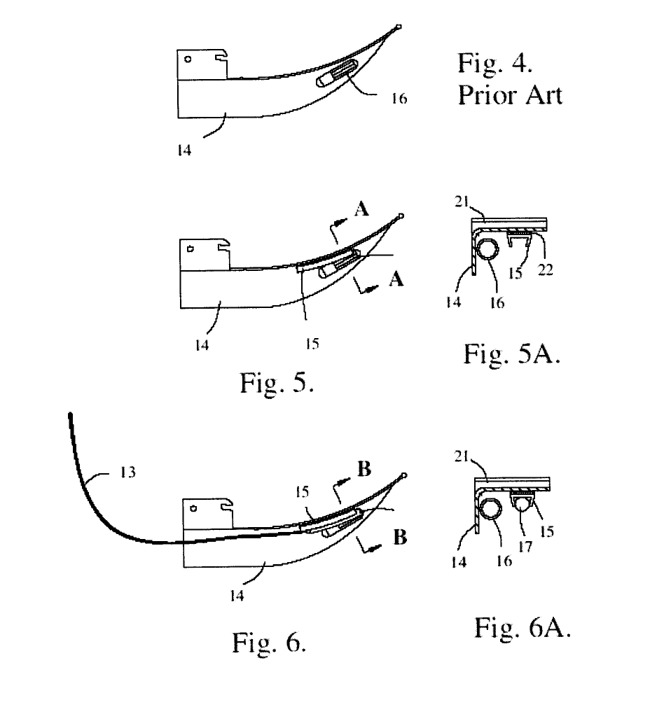

FIG. 4 shows a side view of a conventional curved blade 14. The lamp 16 is integrated into the blade. In FIG. 5, a clip 15, typically made f of nylon or similarly resilient plastic material, is removably attached to the blade 14 near the lamp 16 by means of a pressure sensitive foam strip 22. The foam strip 22 has two surfaces covered with adhesive of different adhesive strength. The adhesive surface attached to the clip 15 has a permanent adhesive, but the adhesive surface attached to the blade has removable adhesive. If the blade 14 has a fiber optic type illuminator, the clip 15 is positioned in a similar place in relation to the light emitting end of the optical fiber bundle.

FIG. 6 shows the video probe 17 held by the clip 15. The distal end of the probe 17 is positioned laterally to the lamp 16.

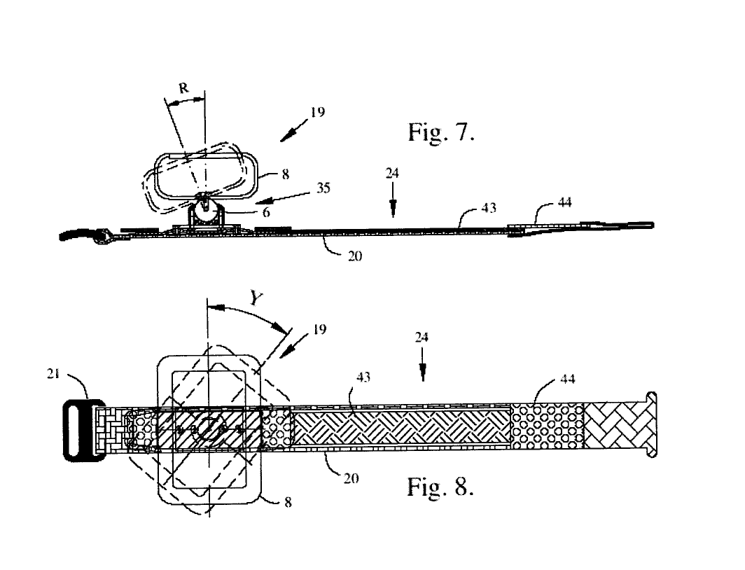

FIGS. 7 and 8 show the video monitor 19 attached to the wrist band 24. The angular adjustment R of the enclosure 8 in vertical plane (roll) is illustrated on FIG. 7, while the angular adjustment Y in horizontal plane (yaw) is shown on FIG. 8. The wrist band 24 has mechanical fastening elements, including a strap portion 20, a buckle 21, hooks and loops areas 43 and 44, for quick securing of the band 24 on the wrist 22. FIG. 10 shows the video monitor 19 attached removably to the wrist band 24 via the pair of reclosable mushroom-type fastener arrays. One array is a permanent part of the foot 4 of the swivel unit 35 and the other array is permanently attached to the plate 25, which is a permanent attached to the wrist band 24.

I claim:

1. A portable video adapter system for attachment to a conventional laryngoscope, the portable video adaptor system comprising: a slim video probe, a bracket that attaches to the blade of the laryngoscope, a portable video monitor electrically connected via flexible wire with the slim video probe, and a means for detachably mounting the video monitor to a wristband or to the handle of the laryngoscope.

2. The portable video adapter system according to claim 1, wherein said means for mounting the video monitor on the wrist includes a swivel unit.

3. The portable video adapter system according to claim 2, wherein the swivel unit includes a pivot joint.

4. The portable video adapter system according to claim 1, wherein the means for detachably mounting the video monitor on an end of the laryngoscope handle includes a swivel unit.

5. The portable video adapter system according to claim 4, wherein the swivel unit includes a ball joint.

6. The portable video adapter system according to claim 5, wherein the ball joint is disjunctive.

7. The portable video adapter system according to claim 1, wherein the means for mounting on a wrist includes a band for wrapping around wrist.

8. The portable video adapter system according to claim 7, wherein the band includes a hook-and-loop fastener.

9. The portable video adapter system according to claim 7, wherein the band includes a platform with a reclosable fastener.

10. The portable video adapter system according to claim 9, wherein the reclosable fastener is an interlocking mushroom-type fastener.

11. The portable video adapter system according to claim 1, wherein the slim video probe includes a CMOS camera and an LED emitting light in front of camera.

12. The portable video adapter system according to claim 1, wherein the LED is a white-type LED.

13. The portable video adapter system according to claim 1, wherein the bracket is a plastic channel having a base, and including a semi-permanent pressure-sensitive adhesive on the external surface of the base.

14. The portable video adapter system according to claim 1, wherein the bracket is a plastic channel having a base, and including a permanent pressure-sensitive adhesive on the external surface of the base.

15. The portable video adapter system according to claim 1, wherein the slim video probe has an elongated curved body, curved along its length to fit the curvature of the laryngoscope blade.

16. The portable video adapter system according to claim 1, wherein the bracket is a plastic channel having a base, and including a semi-permanent pressure-sensitive adhesive on the external and internal surfaces of the base.

17. The portable video adapter system according to claim 1, wherein the bracket is fabricated as a part of the laryngoscope blade.
